(19) United States
(12) Patent Application Publication
(10) Pub. No.: US 2025/0032693 A1
(43) Pub. Date: Jan. 30, 2025

(54) Blood Treatment Apparatus With A Coupled Blood Pump And Compressed Pressure Device

(71) Applicant: Fresenius Medical Care Deutschland GmbH, Bad Homburg (DE)

(72) Inventor: Georg Verch, Bad Homburg (DE)

(21) Appl. No.: 18/717,159

(22) PCT Filed: Dec. 7, 2022
(86) PCT No.: PCT/EP2022/084786

§ 371 Date: Jun. 6, 2024

(30) Foreign Application Priority Data
Dec. 10, 2021 (DE) 10 2021 132 723.7

(51) Int. Cl. A61M 1/36

(52) U.S. Cl. CPC A61M 1/3627; A61M 2205/07; A61M 2205/3334

(57) ABSTRACT

The present disclosure relates to a control device for controlling a blood treatment apparatus when the blood treatment apparatus comprises a compressed air line, a compressed air device being in fluid communication with the compressed air line, a blood pump for conveying blood in a blood tubing set, as well as an arterial patient tube clamp and a venous patient tube clamp. In this, the compressed air device is provided and/or suitable for generating pressure and air flow within the compressed air line. The blood tubing set comprises a venous bubble trap. The control device is configured in order to control or regulate both the blood pump and the compressed air device. The control device is further configured to convey using the blood pump depending on, or as a function of, a conveyance by the compressed air device and/or vice versa.

### CROSS-REFERENCE TO RELATED APPLICATIONS

The present application is the national stage entry of International Application No. PCT/EP2022/084786, filed on Dec. 7, 2022, which claims priority to Application No. DE 10 2021 132 723.7, filed in the Federal Republic of Germany on Dec. 10, 2021, the contents of which are hereby incorporated by reference thereto.

### TECHNICAL FIELD

The present disclosure relates to a control device, a blood treatment apparatus, a digital non-volatile storage medium, a computer program product, and a computer program that relate to a control device for a blood treatment apparatus.

### BACKGROUND

Blood treatment apparatuses are used in practice, the control device of which controls or regulates both a blood pump of the blood treatment apparatus and compressed air devices of the blood treatment apparatus independently of each other.

### SUMMARY

According to the present disclosure, a control device for controlling a blood treatment apparatus is described, wherein the blood treatment apparatus comprises a compressed air line, a compressed air device being in fluid communication with the compressed air line, and a blood pump configured to convey blood all the way through a blood tubing set, within or inside along a blood tubing set comprising a venous bubble trap or air separator when the blood tubing set is connected to the blood treatment apparatus. Further, the blood treatment apparatus comprises an arterial patient tube clamp and a venous patient tube clamp. The compressed air device is provided and/or suitable for generating pressure and/or an air flow within the compressed air line. The compressed air line is provided for being connected to an interior of the venous bubble trap.

The control device is configured to control or regulate both the blood pump and the compressed air device or their conveyance rates.

The control device is further configured to convey using the blood pump depending on, or as a function of, a conveyance or non-conveyance by the compressed air device, and/or vice versa.

According to the present disclosure, there is further described a blood treatment apparatus with a control device according to the present disclosure.

A digital non-volatile storage medium, according to the present disclosure, such as in the form of a machine readable carrier, such as in the form of a diskette, memory card, CD, DVD, EPROM, FRAM (Ferroelectric RAM) or SSD (Solid-State-Drive), particularly with electronically or optically readable control signals, can interact with a programmable computer system, such that a control device is programmed or reprogrammed to be a control device according to the present disclosure and/or such that a blood treatment apparatus is programmed or reprogrammed to be a blood treatment apparatus according to the present disclosure.

A computer program product according to the present disclosure comprises a volatile or transient program code or one stored on a machine readable carrier or a signal wave for prompting the programming or reprogramming of a control device into a control device according to the present disclosure and/or the prompting of the programming or reprogramming of a blood treatment apparatus into a blood treatment apparatus according to the present disclosure. A computer program product may be understood according to the present disclosure as e.g. a computer program stored on a carrier, an embedded system being a comprehensive system with a computer program (e.g., an electronic device with a computer program), a network of computer implemented computer programs (e.g. client/server-system, a cloud computing system etc.), or a computer on which a computer program is loaded, runs, is stored, is executed or developed.

The term “machine readable carrier” as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

A computer program according to the present disclosure encompasses a program code by which the programming or reprogramming of a control device into a control device according to the present disclosure is prompted and/or the programming or reprogramming of a blood treatment apparatus into a blood treatment apparatus according to the present disclosure is prompted when the computer program runs on a computer, respectively.

Embodiments according to the present disclosure may comprise one or more of the following features in any combination, unless the person skilled in the art recognizes their combination as technically impossible.

In all of the statements made herein, the use of the expression “may be” or “may have” and so on, is to be understood synonymously with “preferably is” or “preferably has,” and so on respectively, and is intended to illustrate embodiments according to the present disclosure.

Whenever numerical words are mentioned herein, a person skilled in the art would recognize or understand them as indications of a numerical lower limit. Unless stated otherwise, the person skilled in the art would comprehend the specification for example of “one” (also “a/an”) as encompassing “at least one”. This understanding is also equally encompassed by the present disclosure as the interpretation that a numeric word, for example, “one” (also “a/an”) may alternatively mean “exactly one”, wherever this is evidently technically possible for the person skilled in the art. Both understandings are encompassed by the present disclosure and apply herein to all used numerical words.

Whenever reference is made herein to spatial indications, such as “top”, “bottom”, “left” or “right”, the skilled person understands this to mean the arrangement in the figures attached herein and/or in the state of use. “Bottom” is closer to the center of the earth or the lower edge of the figure than “top”.

Advantageous developments of the present disclosure are each also subject-matter of the dependent claims and embodiments.

Whenever an embodiment is mentioned herein, it is then an exemplary embodiment according to the present disclosure.

When it is disclosed herein that the subject-matter according to the present disclosure comprises one or several features in a certain embodiment, it is also respectively disclosed herein that the subject-matter according to the present disclosure does, in other embodiments, likewise according to the present disclosure, explicitly not comprise this or these features, for example, in the sense of a disclaimer. Therefore, for every embodiment mentioned herein it applies that the converse embodiment, e.g. formulated as negation, is also disclosed.

Whenever programed or configured is mentioned herein, then these terms may in some embodiments be interchangeable.

When reference is made herein to a signal communication or communication connection between two components, this may be understood to mean a connection that exists in use. Likewise, it may be understood herein that there is a preparation for such a signal communication (wired, wireless, or otherwise implemented), for example, by a coupling of both components, such as by pairing, etc.

Pairing is to be understood as a process that takes place in connection with computer networks in order to establish an initial link between computer units for the purpose of communication. One example of this is the establishment of a Bluetooth connection, by which various devices (e.g. smartphone, headphones) are connected to one another. Pairing is sometimes also referred to as bonding.

In several embodiments, the control device is present in or on the blood treatment apparatus, for instance together with other components or devices of the blood treatment apparatus in a common housing of the blood treatment apparatus.

In some embodiments, the dependency consists of, or encompasses, not allowing, preventing, or, in particular actively automatically terminating a conveying by the compressed air device during the conveying by the blood pump, or when the blood pump starts conveying. This form of dependency is also referred to herein as safety condition 1.

If safety condition 1 is met or fulfilled, it may advantageously be ensured that the venous flow rate for blood into the patient's vascular system, e.g., during blood return, does not exceed the blood flow rate of the blood pump or the maximum permissible effective blood flow rate for the respective type of therapy and/or patient weight class, e.g., the return rate ultimately resulting for the patient, which, due to the effect of the compressed air device on the blood located downstream of the blood pump in the extracorporeal blood circuit, e.g., in the blood tubing set, does not exceed the desired blood flow rate set for the blood pump or does not exceed a maximum permissible effective blood flow rate for the respective type of therapy and/or patient weight class.

The safety condition 1 is realized, for example, by the compressed air device, e.g. the compressor, being switched off and locked optionally in two channels via the operating system or the control device and/or directly via a second switch-off path.

Thus, if the control device is configured to convey by the compressed air device depending on, or as a function of, a conveyance by the blood pump, this may mean that the control device is programmed and/or configured to take into account, when conveying by the compressed air device, that, or to what extent, the blood pump is already conveying or will convey. Alternatively, it may mean that the compressed air device cannot or must not convey if the blood pump is already conveying. This can be ensured by the control device.

Conversely, e.g., if the control device is configured to convey by the blood pump depending on, or as a function of, a conveyance by the compressed air device, this may mean that the control device is programmed and/or configured to take into account, when conveying by the blood pump, that, or to what extent, the compressed air device is already conveying or will convey. Alternatively, it may mean that the blood pump cannot or must not convey if the compressed air device is already conveying. This can also be ensured by the control device.

In several embodiments of the control device according to the present disclosure, the dependency consists of, or encompasses, not allowing, preventing, or, in particular, actively terminating a conveyance by the blood pump during the conveyance by the compressed air device. This form of dependency is also referred to herein as safety condition 2.

In some embodiments of the control device according to the present disclosure, the dependency consists in limiting a conveyance of a fluid simultaneously by the blood pump and indirectly via compressed air by the compressed air device, which impacts or acts on in a conveying manner on, e.g., a blood level in the bubble trap, in such a way that a predetermined maximum conveyance rate is not exceeded. If the blood pump conveys the fluid, for example, at 0.8×ml/min at a maximum conveying rate of x ml/min, then a controlling of the compressed air device may consist of allowing the compressed air device to convey compressed air to a maximum extent such that the effect of the compressed air on the blood does not result in any further conveying of the blood than a maximum of 0.2×ml/min. Thus, also in this case, a conveyance by the compressed air device is given in dependence on an (already given) conveyance by the blood pump, whereas the reverse case, in which conveyance by the blood pump is given in dependence on the conveyance by the compressed air device, is also encompassed by the present disclosure.

An operation of the compressed air device may lower the liquid level (blood) in the bubble trap, the so-called “level lowering”, and may take place during an opening of a pressure measuring unit, a coupling test of a pressure measuring unit or a recoupling of a pressure measuring unit.

During the “level lowering”, the compressed air device operates throttled such that the flow rate of the blood flowing extracorporeally towards the venous return line cannot or can never exceed a predefined upper limit value, set to 250 ml/min in the exemplary embodiment of a pediatric extracorporeal blood treatment, so that the flow rate in the area of the air bubble detector (ABD) can never exceed the upper limit value of e.g. 250 ml/min. If the compressed air device is already conveying, it may be provided to completely prevent conveying by the blood pump.

Regardless of the specific control of blood pump and/or compressed air device, the control device may be programmed to issue an alarm when the limits of the maximum flow rate for detecting individual air bubbles, continuous air infusion, or microbubbles are exceeded.

In some embodiments of the control device, the latter is configured to allow the arterial patient tube clamp and/or the venous patient tube clamp to close, or to remain closed, during a conveyance by the compressed air device which takes place with at least 15%, preferably at least 20%, of its maximum conveying rate and/or which takes place unthrottled. This is also referred to herein as safety condition 3.

An operation, defined as above, e.g. unthrottled, of the compressed air device, e.g. of the compressor, takes place e.g. during the opening of a pressure measuring unit, a coupling test of a pressure measuring unit, a new coupling of a pressure measuring unit, to which a predetermined pressure, e.g. the maximum pressure (about 2 bar), must be built up.

The control device may be configured to automatically keep the access and return clamps on the device side closed or to have them closed already when the compressed air device is conveying with more than 15% of its maximum capacity.

When it is mentioned here that two processes—for instance conveying by the blood pump as one process and conveying by the compressed air line as another process-take place at the same time, or when it is mentioned that one state should occur or be maintained if or during a second state or process begins, this includes that both processes or states begin, exist and/or end at the same time. However, it is also encompassed that they overlap, for example that one state is terminated when, as soon as and/or because another process or state begins or occurs. Thus, it may for example be provided to terminate the operation of the blood pump at, before or after a point in time at which conveying by the compressed air device takes place, or at which conveying should start. In some embodiments, it is thus not so much the time at which another process occurs that is relevant, but rather that the other process is initiated with regard to the process that is already in progress.

Terminating a conveyance may be understood herein as e.g. calling off a conveyance rate of 0 ml/min or 0 ml/see or e.g. not continuing to build up pressure which is already present, using the relevant conveying device.

In several embodiments of the blood treatment apparatus according to the present disclosure, the blood treatment apparatus is operatively connected to a blood tubing set comprising a venous bubble trap. The compressed air line of the blood treatment apparatus may be in fluid communication with the venous bubble trap.

In certain embodiments, the blood tubing set may be a blood tubing set for pediatric treatment.

In some embodiments, the blood treatment apparatus and/or the blood tubing set are connected to a blood filter or dialyzer for pediatric blood treatment.

In several embodiments, the blood treatment apparatus and/or the blood tubing set are connected to a blood filter or dialyzer for adult blood treatment.

In some embodiments, the blood treatment apparatus is embodied as an apparatus for apheresis or dialysis, again in particular for hemodialysis, hemofiltration, hemodiafiltration.

In several embodiments, the blood treatment apparatus is embodied as an apparatus for the acute renal replacement therapy, the chronic renal replacement therapy or the continuous renal replacement therapy (CRRT=continuous renal replacement therapy).

Some or all embodiments according to the present disclosure may have one, several or all of the advantages mentioned above and/or below.

An advantage of the present disclosure may be that patient safety is improved in the event of an error, particularly in pediatric extracorporeal blood treatment procedures, by (or on the basis or with the aid of) the new restrictive safety conditions 1, 2 and/or 3 in the programming of the control device of the blood treatment apparatus according to the present disclosure, by the herein described new restrictive safety.

It may, thus, be advantageously ensured that the venous flow rate for blood into the patient's vascular system, e.g., during blood return, does not exceed the blood flow rate of the blood pump. The return rate ultimately resulting for the patient, due to the effect of the compressed air device on the blood present downstream of the blood pump in the blood tubing set, is thus reliably not above the desired blood flow rate set for the blood pump. This advantageously increases patient safety by preventing the treated blood from being reinfused into the patient's body at too high a pressure and/or flow rate.

By using the present disclosure, it may advantageously be achieved that the resulting return rate is not above the actual blood flow rate set for the blood pump (which may also be set very low). Advantageously, it can also be achieved that the resulting return rate is not above a maximum allowable effective blood flow rate for the particular type of therapy and/or patient weight class. In one example of pediatric treatment, e.g. the blood flow rate should not exceed 250 ml/min. For this reason, in such embodiments, the maximum adjustable blood flow rate at the pump may be only 200 ml/min.

Another advantage of the present disclosure may, in some embodiments, be that an unintentional supply of air into the bubble trap is avoided. Thus, for example in the event of an error in the switch valve and/or switch valve control, air that is provided for opening one of the further pressure measuring units could inadvertently get into the bubble trap. It is hardly possible to check the correct function of the switch valve when the blood treatment apparatus is in use, e.g., under certain circumstances it is not guaranteed that the switch valve actually only supplies this air to the pressure measuring unit so that the switch valve may be opened. In such an error event, air would also be directed into the bubble trap, which in turn could lead to increased blood flow towards the patient. This is advantageously avoided by means of the present disclosure and thus patient safety is increased.

Another advantage of the present disclosure may, in several embodiments, be that a harmful effect of the compressed air device on the blood tubing set, and above all on the pediatric blood tubing set, is prevented by the blood tubing set being protected against the undesired or impermissible effect of the compressed air device in that in some embodiments the clamps are automatically closed when or as long as the compressed air device conveys e.g. unthrottled.

It is also advantageous that the present disclosure may also be implemented in blood treatment apparatuses that have already been delivered and are in use, by simple software updates. Retrofitting of existing systems is thus possible without significant effort.

### BRIEF DESCRIPTION OF THE DRAWINGS

In the following, the present disclosure is exemplarily described with reference to the accompanying figures in which identical reference numerals designate same or similar components. The following applies:

FIG. 1 shows schematically simplified sections of a blood treatment apparatus according to the present disclosure as well as sections of a blood tubing set connected to the blood treatment apparatus;

FIG. 2 shows schematically simplified a flow diagram of a continuous veno-venous hemodiafiltration (CVVHDF) with post-dilution of a blood treatment apparatus according to the present disclosure in a first exemplary embodiment; and

FIG. 3 shows schematically simplified a fluid line structure of a blood treatment apparatus according to the present disclosure in a further exemplary embodiment.

### DETAILED DESCRIPTION

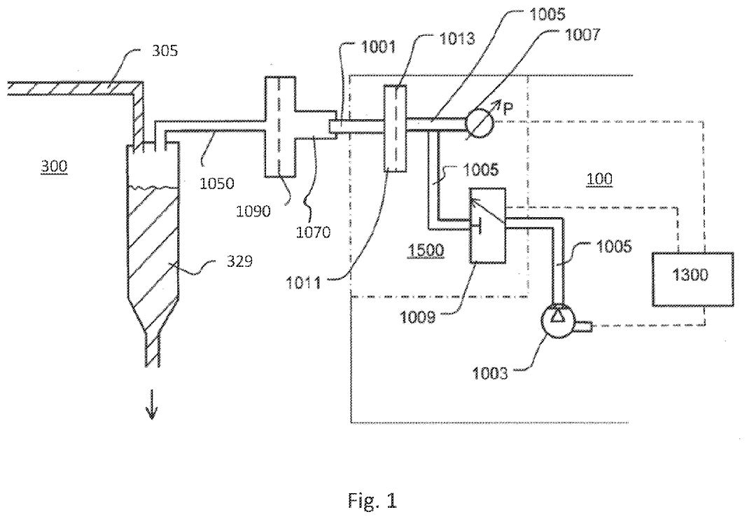

FIG. 1 shows schematically very simplified sections of a blood treatment apparatus 100 according to the present disclosure as well as sections of a blood tubing set 300 or an extracorporeal blood circuit connected to the blood treatment apparatus 100.

The blood tubing set 300, which may extend optionally outside and within a blood cassette, not shown, comprises a venous patient line 305 and a venous bubble trap 329. A flow through the patient line 305 may, when used in the direction of the arrow which is indicated downstream of the blood filter 303 or dialyzer, take place toward the patient.

A blood tubing set 300 is connected to a pressure measuring line 1050, which in FIG. 1 extends from the bubble trap 329 in a exemplary manner. The pressure measuring line 1050, which leads from the bubble trap 329 to the pressure sensor PS3, may be the return pressure measuring line. The pressure measuring line 1050 may be part of the blood tubing set 300 or part of the blood treatment apparatus 100.

The pressure measuring line 1050 comprises herein optionally a connector 1070 which is provided and designed for connecting the pressure measuring line 1050 to a compressed air outlet 1001 of the blood treatment apparatus 100. Optionally, the connector 1070 and the compressed air outlet 1001 are female or male halves of a Luer connector having a female or male sealing cone or of a corresponding Luer lock connector having an additional locking thread.

The compressed air outlet 1001 may be disposed in or on an outer wall of the blood treatment apparatus 100, for example in a housing wall of the blood treatment apparatus 100.

The connector 1070 of the pressure measuring line 1050, or another section of the pressure measuring line 1050, which is disposed in an area of the pressure measuring line 1050 through which air flows or through which air may flow during use of the pressure measuring line 1050, preferably comprises an air-permeable membrane 1090. The air-permeable membrane 1090 is designed, for example, as a hydrophobic air-permeable membrane or hydrophobic filter.

The blood treatment apparatus 100 comprises a compressor 1003 as an example of a compressed air device or compressed air source.

Compressor 1003 (also referred to herein as compressed air device 1003) and compressed air outlet 1001 are connected in fluid communication by a compressed air line 1005. The compressor 1003 may optionally comprise further valves for use other than those described herein.

In or on the compressed air line 1005, preferably a pressure sensor 1007 and, optionally, a switch valve 1009 (alternatively or additionally a throttle, a switch, a lock and/or the like) are provided.

As FIG. 1 shows, the pressure sensor 1007 is preferably integrated into the compressed air line 1005 or is in suitable fluid communication therewith such that the pressure sensor 1007 can measure the pressure P prevailing in the pressure line 1005 due to the operation of the compressor 1003 when the switch valve 1009 (if present) is switched accordingly and the compressor 1003 is active, e.g., switched on. The pressure sensor 1007 may be the return pressure sensor.

In example implementations, the compressed air line 1005 leads through an optionally provided protection filter 1011, which in turn comprises a, preferably hydrophobic, air-permeable membrane 1013 located in the flow path. If such an air-permeable membrane 1013 is provided, the detection device 1300 may optionally be configured or programmed to take corrective account of the pressure resistance represented by the air-permeable membrane 1013 (for example by filtering, subtracting, etc.).

The blood treatment apparatus 100 preferably comprises the detection device 1300. As shown by dashed lines, this is exemplarily connected to the compressor 1003, the pressure sensor 1007 and/or the switch valve 1009 in signal communication.

As shown by dash-dotted lines, at least the compressed air outlet 1001 and the pressure sensor 1007, further optionally the switch valve 1009 and the protection filter 1011, if present, may be part of the independent pressure measuring unit 1500, which is connected to the blood treatment apparatus 100.

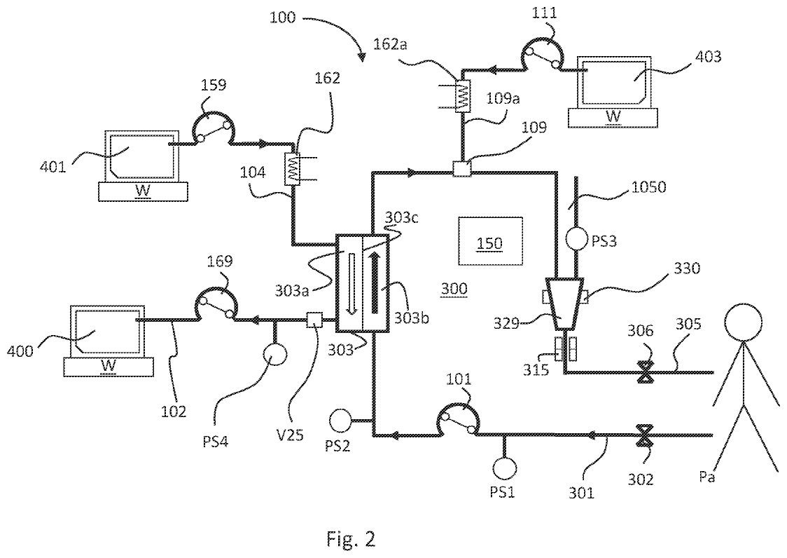

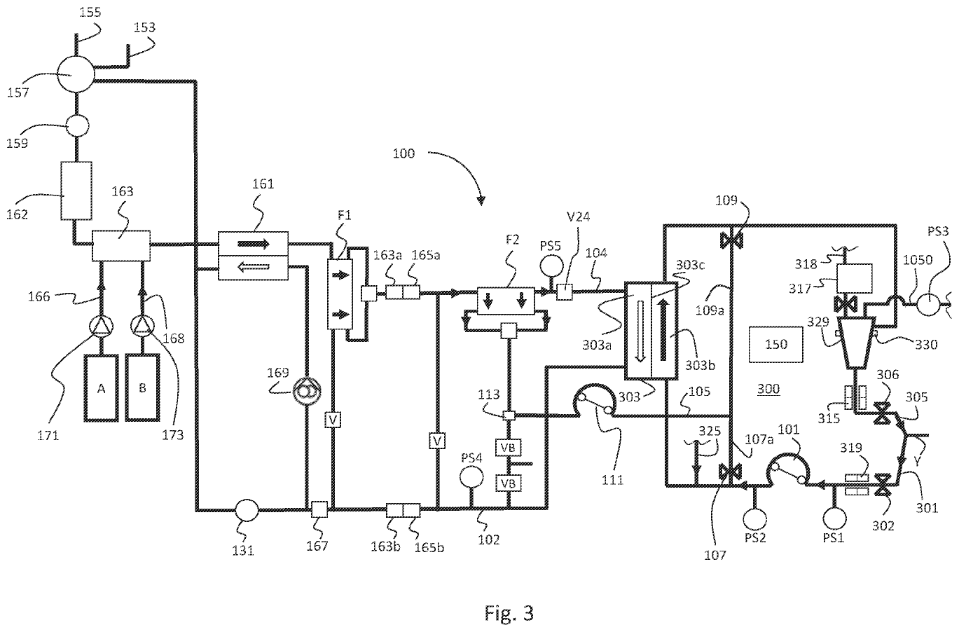

By using the switch valve 1009, the air can be directed, for example, either towards the venous pressure measuring line 1050 of the bubble trap 329 or towards one of the further pressure measuring units, for example towards the pressure sensors for arterial pressure PS1, PS2, towards the venous pressure sensor PS3, towards the pressure sensor for measuring the filtrate pressure PS4 (not shown in FIG. 1, see FIG. 2 and FIG. 3). The pressure measuring units are each set up for coupling a pressure dome, on the disposable side, to the respective pressure sensor. Opening each of the mentioned pressure measuring units for the purpose of inserting the pressure dome takes place via compressed air from the compressed air device 1003, here the compressor.

The mentioned pressure measuring units PS1, PS2, PS4 are, for example, of the type described in patent application WO 2011/015309 A1.

FIG. 2 shows a schematically very simplified flow diagram of a continuous veno-venous hemodiafiltration (CVVHDF) with post-dilution of a blood treatment apparatus according to the present disclosure in a first exemplary embodiment.

The blood treatment apparatus 100 according to the present disclosure (shown in FIG. 2 only by individual, schematically highly simplified components) comprises a blood pump 101 for conveying blood through a blood tubing set of an extracorporeal blood circuit 300, which in turn comprises a bubble trap 329.

The blood tubing set 300 has an arterial line section 301 (also referred to as first line, arterial patient line, or blood withdrawal line) interacting with an arterial patient line clamp 302 and said blood tubing set 300 may be closed by the latter. The blood tubing set 300 further comprises, or is connected to, connectors for an arterial connection needle (not shown in FIG. 2) or for an arterial connection of a central venous catheter. Further, the blood tubing set 300 may be closed at a venous line section 305 (also referred to as venous patient line, blood return line, or second line) by a venous patient tube clamp 306, and has, or is connected to, connectors for a venous connection needle (not shown in FIG. 2) or for a venous connection of a central venous catheter.

In the example of FIG. 2, an embodiment of a control device 150 according to the present disclosure may be seen, which is configured to control or regulate the blood treatment apparatus 100, in particular (to control or regulate) both the blood pump 101 and the compressed air device 1003.

The blood treatment apparatus 100 is in fluid communication with a compressed air device 1003, such as a compressor (see FIG. 1) via, amongst others, the pressure measuring line 1050. In some embodiments, the compressed air device 1003 is used to generate pressure and a flow of air within the compressed air line 1005 toward the bubble trap 329.

The control device 150 may further be configured to convey by using the blood pump 101, in particular depending on, or as a function of, a conveyance by the compressed air device 1003. Alternatively or additionally, the control device 150 may be configured to convey by using the compressed air device 1003, in particular depending on, or as a function of, a conveyance by the blood pump 101. With regard to the possible configuration of the control device 150, reference is made to statements made elsewhere herein.

The blood tubing set 300 or the extracorporeal blood circuit comprises, in the example of FIG. 2, further a blood filter 303 which blood chamber 303b is connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, e.g., spent dialysis liquid, leading away from the dialysis liquid chamber 303a. For this purpose, suitable connectors are used on the dialysis liquid inlet line 104 or on the dialysate outlet line 102 on the one hand and on the dialysate ports on the other hand, which may be connected to each other, in particular releasably.

Dialysis liquid chamber 303a and blood chamber 303b are separated from each other by a mostly semi-permeable membrane 303c. Blood and dialysis liquid are mostly guided through the blood filter 303 by the counter current principle. The blood is purified in the blood filter 303.

In the example of FIG. 2, a first flow pump 159 is used to convey dialysis liquid from a dialysis liquid source 401, here a bag with dialysis liquid, to the dialysis liquid chamber 303a via a dialysis liquid inlet line 104.

The dialysate, which is also referred to as effluent and which is, or comprises, spent dialysis liquid, optionally enriched with filtrate, leaves the dialysis liquid chamber 303a of the blood filter 303 via the dialysate outlet line 102, optionally conveyed by a second flow pump 169. In the example of FIG. 2, the effluent is first collected in an optional effluent bag 400 to be subsequently discarded into a drain.

The scales W are used to determine the amount of dialysis liquid supplied or the amount of effluent (also: filtrate) discharged. The scales W, or their measurements values, are used for balancing.

Using the postdilution valve 109, the blood tubing set 300 is supplied with substitute fluid from a substitute fluid source 403, herein a substitute fluid bag. This is conveyed by a substitute fluid pump 111 arranged in or on the line 109a associated with the post-dilution valve; optionally the substitute fluid is heated or brought to a predetermined temperature in a heating device 162a. By using the scale W, the supplied amount of substitute fluid may be determined. The values provided by the scale W may also be used for balancing.

The blood pump 101, when lowering an excessive blood level in the bubble trap 329, which may have been detected by a level detector 330, mostly runs forward, e.g., with the usual direction of rotation during a blood treatment. The lowering of an excessively high blood level in the bubble trap 329 during treatment with a connected patient Pa is mostly done by building up an overpressure on the surface of the liquid in the bubble trap 329 by forcing air into the blood-free space of the bubble trap 329 from above using the compressed air device 1003.

If the compressed air device 1003 is thereby conveying, there may be a calculated flow across the air bubble detector 315 that could render difficult a correct air bubble detection at the air bubble detector.

The blood pump 101 generates a certain flow rate in the blood tubing set 300. If the level in the bubble trap 329 is constant, the venous blood flow rate into the patient Pa is equal to the blood flow rate of the blood pump 101. If the level in the bubble trap 329 rises, the venous blood flow rate in the patient Pa may be correspondingly lower than the blood flow rate of the blood pump 101.

If the compressed air device 1003 is controlled, by e.g. the user in the Graphical User Interface (GUI) by the “level rocker” icon, in order to lower the level in the bubble trap 329, an additional flow rate from the bubble trap 329 into the venous line section 305 (venous return line) is generated, e.g., the flow rate from the bubble trap 329 and the blood flow rate from the blood pump 101 add up, which could cause the venous blood flow rate into the patient Pa to exceed the blood flow rate from the blood pump 101, which, particularly in the event of a failure, could cause critical flow rates into the patient Pa.

In particular, this could pose a risk to pediatric patients Pa. In particular, if blood flow rates are too high, eventual air bubbles/microbubbles present in the blood cannot be reliably detected by the air bubble detector 315. This scenario may be excluded with respect to the elaborated dependencies above.

FIG. 3 shows, schematically simplified, a fluid line structure of a blood treatment apparatus 100 in an exemplary embodiment.

The blood treatment apparatus 100 is connected to a blood tubing set 300, which may be connected to the vascular system of the patient, not shown, for executing a treatment using double-needle access, or using e.g. an additional Y-connector (reference numeral Y), as shown in FIG. 3 using single-needle access. The blood tubing set 300 may be present, optionally in sections thereof, in or on a blood cassette.

Pumps, actuators and/or valves in the area of the blood tubing set 300 are connected to the blood treatment apparatus 100 according to the present disclosure or to a control device 150 according to the present disclosure.

The blood tubing set 300 comprises (or is connected to) an arterial patient tube clamp 302 and an arterial connection needle of an arterial section or of an arterial patient line, blood withdrawal line or first line 301 or an arterial connection of a central venous catheter. The blood tubing set 300 also comprises (or is connected to) a venous patient tube clamp 306 and a venous connection needle of a venous section, a venous patient line, a blood return line or a second line 305 or a venous connection of a central venous catheter.

A blood pump 101 is provided in or at the first line 301, a substituate pump 111 is connected to a dialysis liquid inlet line 104 for conveying fresh dialysis liquid, which is filtered in a further filter stage F2 (substitute fluid). A substitute fluid line 105 may be fluidically connected to the inlet line 104. Using the substitute fluid pump 111, substitute fluid may be introduced by predilution, via a predilution valve 107, or by postdilution, via a postdilution valve 109, via associated lines 107a or 109a into line sections, for example into the arterial line section 301 or into the venous line section 305 (here between a blood chamber 303b of a blood filter 303 and a venous air separation chamber or a bubble trap 329) of the blood tubing set 300.

The blood filter 303 comprises the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, e.g., spent dialysis liquid, leading away from the dialysis liquid chamber 303a. Suitable connectors, which may be connected to each other, in particular releasably, disposed on the dialysis liquid inlet line 104 or on the dialysate outlet line 102 on one hand and on the dialysate port on the other hand, serve for this purpose.

Dialysis liquid chamber 303a and blood chamber 303b are separated from each other by a mostly semi-permeable membrane 303c. The semi-permeable membrane 303c represents the separating borderline between the blood side with the extracorporeal blood circuit 300 and the machine side with the dialysis liquid circuit or the dialysate circuit, respectively, which is shown in FIG. 3 to the left of the membrane 303c.

The bubble trap 329 may, as shown in FIG. 1, be in fluid communication with the compressed air device 1003 via the pressure measuring line 1050.

The arrangement of FIG. 3 further encompasses a valve V24 arranged in the dialysis liquid inlet line 104 upstream of the blood filter 303, but downstream of a first pressure sensor PS5.

The arrangement in FIG. 3 encompasses an optional air bubble detector 315 for detecting air and/or blood. The arrangement of FIG. 3 further encompasses one or two pressure sensors PS1 (upstream of the blood pump 101) and PS2 (downstream of the blood pump 101, which measures the pressure upstream of the blood filter 303 (“pre-hemofilter”)) at the points shown in FIG. 3. Further pressure sensors may be provided, e.g. pressure sensor PS3 downstream of the venous bubble trap 329

An optional single-needle chamber 317 is used in FIG. 2 as a buffer and/or compensating reservoir in a single-needle procedure in which the patient is connected to the extracorporeal blood tubing set 300 using only one of the two blood lines 301, 305.

The arrangement of FIG. 3 also encompasses an optional detector 319 for detecting air bubbles and/or blood.

An optional Heparin addition site 325 may be provided.

On the left in FIG. 3, a mixing device 163 is shown, which provides a predetermined mixture for the respective solution from the containers A (for A concentrate via concentrate supply 166) and B (for B concentrate via concentrate supply 168) for use by the blood treatment apparatus 100. The solution contains water from the water source 155 (on-line, e.g. as reverse osmosis water or from bags), wherein the water is heated e.g. in the heating device 162.

A pump 171, which can be referred to as concentrate pump or sodium pump, is fluidly connected to the mixing device 163 and a source of sodium, for example the container A, and/or coveys out of it. An optional pump 173, which is assigned to container B, for example for bicarbonate, can be seen.

Furthermore, FIG. 3 shows a drainage 153 for the effluent. An optional heat exchanger 157 and a first flow pump 159, which is suitable for degassing, complete the arrangement shown.

The pressure sensor PS4 may be provided downstream of the blood filter 303 on the water side, but preferably upstream of the ultrafiltration pump 131 in the dialysate outlet line 102 for measuring the filtrate pressure or membrane pressure of the blood filter 303.

Blood leaving the blood filter 303 flows through an optional bubble trap 329, which may comprise a deaeration device 318 and may be in fluid communication with the pressure sensor PS3.

The exemplary arrangement shown in FIG. 3 comprises a control device 150, which may be in a wired or wireless signal connection with any of the components mentioned herein-especially or in particular with the blood pump 101—to control or regulate the blood treatment apparatus 100

By using the device for on-line mixing of the dialysis liquid, a variation of sodium content, controlled by the control device 150, is possible within certain limits. For this purpose, in particular the measured values determined by the conductivity sensors 163a, 163b may be taken into account. Should an adjustment of the sodium content of the dialysis liquid (sodium concentration) or of the substituate turn out to be necessary or desired, this can be done by adjusting the conveyance rate of the sodium pump 171.

In addition, the blood treatment apparatus 100 comprises means for conveying fresh dialysis liquid and dialysate. A first valve may be provided between the first flow pump 159 and the blood filter 303, which first valve opens or closes the inflow towards the blood filter 303 at the inlet side. A second, optional flow pump 169 which conveys dialysate through the drainage line 153 is provided e.g. downstream of the blood filter 303. A second valve may be provided between the blood filter 303 and the second flow pump 169 at the outlet side, which second valve opens or closes the outflow.

Furthermore, the blood treatment apparatus 100 optionally comprises a device 161 for balancing the flow flowing into and out of the dialyzer 303 on the machine side. The device 161 for balancing is preferably arranged in a line section between the first flow pump 159 and the second flow pump 169.

The blood treatment apparatus 100 further comprises means, such as the ultrafiltration pump 131, for the precise removal of a volume of liquid, as predetermined by the user and/or by the control device 150, from the balanced circuit.

Sensors such as the optional conductivity sensors 163a, 163b serve to determine the conductivity, which in some embodiments is temperature-compensated, as well as the fluid flow upstream and downstream of the dialyzer 303.

Temperature sensors 165a, 165b may be provided as one or a plurality thereof. Temperature values supplied by them may be used to determine a temperature-compensated conductivity.

A leakage sensor 167 is optionally provided, and may be provided at or in a port of the blood treatment apparatus 100.

Further flow pumps in addition or alternatively to e.g. the one with the reference numeral 169 may also be provided.

A number of optional valves are each denoted with V in FIG. 3; by-pass valves with VB.

In several embodiments, the control device 150 determines the electrolyte and/or liquid balance based on the measured values of the aforementioned optional sensors.

Filters F1 and F2 can be provided, connected in series.

Even when using non-pure water, the filter F1 exemplarily serves herein to generate sufficiently pure dialysis liquid by the mixing device 163, which then flows through the blood filter 303, e.g. using the countercurrent principle.

The filter F2 exemplarily serves herein to generate sterile or sufficiently filtered substitute fluid from the sufficiently pure dialysis liquid leaving the first filter F1, by filtering e.g. pyrogenic substances. This substituate may then be safely added to the extracorporeally flowing blood of the patient and thus ultimately to the patient's body.

The blood treatment apparatus 100 is optionally shown in FIG. 3 as a device for hemo(dia) filtration. However, hemodialysis apparatuses are also covered by the present disclosure, although not specifically represented in a figure.

The present disclosure is not limited to the embodiments described supra; they only serve for illustration.

The arrows shown in the figures generally indicate each the flow direction.

### LIST OF REFERENCE NUMERALS

100 blood treatment apparatus
101 blood pump
102 dialysate outlet line
104 dialysis liquid inlet line
105 substitute fluid line
107 pre-dilution valve
107a line associated with the pre-dilution valve
109 post-dilution valve
109a line associated with the post-dilution valve
111 substitute fluid line
131 ultrafiltration pump
150 control device
153 drainage or discharge
155 water source
157 heat exchanger
159 first flow pump
161 device for balancing
162 heating device
162a heating device
163 mixing device
163a conductivity sensor
163b conductivity sensor
165a temperature sensor
165b temperature sensor
166 concentrate supply
167 leak sensor
168 concentrate supply
169 second flow pump
171 pump, sodium pump
173 pump, bicarbonate pump
300 extracorporeal blood tubing set or blood circuit
301 first line (arterial line section)
302 arterial patient tube clamp
303 blood filter or dialyzer
303a dialysis liquid chamber
303b blood chamber
303c semi-permeable membrane
305 second line (venous line section)
306 venous patient tube clamp
315 air bubble detector; ABD
317 single-needle chamber
318 deaeration device
319 detector
325 addition site for Heparin
329 venous bubble trap or air separator
330 filling level detector
400 effluent bag; filtrate bag
401 dialysis liquid source;
bag with dialysis liquid
403 substitute fluid source; bag with substitute fluid
1001 compressed air outlet or exit
1003 compressed air device, e.g. compressor
1005 compressed air line
1007 pressure sensor
1009 switch valve
1011 protection filter
1013 air-permeable membrane
1050 pressure measuring line
1070 connector
1090 air-permeable membrane
1300 detection device
1500 pressure measuring unit
A container; A-concentrate; sodium
B container; B-concentrate; bicarbonate
F1 filter
F2 filter
K compressed air source, compressor
Pa patient
PS1 arterial pressure sensor (optional)
PS2 arterial pressure sensor (optional)
PS3 pressure sensor (optional)
PS4 pressure sensor for measuring the filtrate pressure (optional)
PS5 pressure sensor for measuring the pressure in the dialysis liquid inlet
line
V valves
V24 valve
VB bypass valve
W scale
Y Y-connector

1-12. (canceled)

13. A control device configured for controlling a blood treatment apparatus, wherein:
the blood treatment apparatus comprises:
a compressed air line;
a compressed air device in fluid communication with the compressed air line, the compressed air device configured for generating pressure and/or an air flow within the compressed air line;
a blood pump for conveying blood through or within a blood tubing set that comprises a venous bubble trap;
an arterial patient tube clamp; and
a venous patient tube clamp;
the control device is configured to control or regulate both the blood pump and the compressed air device; and
the control device is further configured (i) to convey using the blood pump based on a conveyance by the compressed air device or (ii) to convey using the compressed air device based on a conveyance by the blood pump.

14. The control device according to claim 13, wherein the control device is configured for disallowing, preventing, limiting to a predetermined value, or terminating a conveyance by the compressed air device during conveyance by the blood pump.

15. The control device according to claim 13, wherein the control device is configured for disallowing, preventing, limiting to a predetermined value, or terminating a conveyance by the blood pump during conveyance by the compressed air device.

16. The control device according to claim 13, wherein, during conveyance by the compressed air device with at least 15% of a maximum conveying capacity of the compressed air device, the arterial patient tube clamp and/or the venous patient tube clamp closes or remains closed.

17. The control device according to claim 13, wherein during conveyance by the compressed air device with at least 20% of a maximum conveying capacity of the compressed air device, the arterial patient tube clamp and/or the venous patient tube clamp closes or remains closed.

18. A blood treatment apparatus comprising:
a compressed air line;
a compressed air device in fluid communication with the compressed air line, wherein the compressed air device is configured for generating pressure and/or an air flow within the compressed air line;
a blood pump for conveying blood through or within a blood tubing set that comprises a venous bubble trap;
an arterial patient tube clamp;
a venous patient tube clamp; and
a control device configured to control or regulate both the blood pump and the compressed air device, wherein the control device is further configured (i) to convey using the blood pump based on a conveyance by the compressed air device or (ii) to convey using the compressed air device based on a conveyance by the blood pump.

19. The blood treatment apparatus according to claim 18, wherein the blood treatment apparatus is connected to the blood tubing set that comprises the venous bubble trap.

20. The blood treatment apparatus according to claim 18, wherein the blood treatment apparatus is connected to a blood filter or dialyzer for pediatric blood treatment.

21. The blood treatment apparatus according to claim 18, wherein the blood treatment apparatus is connected to a blood filter or dialyzer for adult blood treatment.

22. The blood treatment apparatus according to claim 18, wherein the blood treatment apparatus is configured for apheresis or dialysis.

23. The blood treatment apparatus according to claim 18, wherein the blood treatment apparatus is configured for hemodialysis, hemofiltration, or hemodiafiltration.

24. The blood treatment apparatus according to claim 18, wherein the blood treatment apparatus is configured for acute renal replacement therapy, chronic renal replacement therapy, or continuous renal replacement therapy (CRRT).

25. A non-transitory digital storage medium with electronically readable control signals configured to interact with a programmable computer system to program or reprogram the control device according to claim 13.

26. A non-transitory digital storage medium with electronically readable control signals configured to interact with a programmable computer system to program or reprogram the blood treatment apparatus according to claim 18.

27. A computer program product configured to interact with a programmable computer system to program or reprogram the control device according to claim 13.

28. A computer program product configured to interact with a programmable computer system to program or reprogram the blood treatment apparatus according to claim 18.

29. A computer program having a program code for programming or reprogramming the control device according to claim 13.

30. A computer program having a program code for programming or reprogramming the blood treatment apparatus according to claim 18.
